(19) United States
(12) Patent Application Publication
(10) Pub. No.: US 2025/0262135 A1
(43) Pub. Date: Aug. 21, 2025

(54) EMULSION WITH LIQUID CRYSTALLINE STRUCTURES

(71) Applicant: BEIERSDORF AG, Hamburg (DE)

(72) Inventors: Wei TANG, Hamburg (DE); Kerstin FRANCK, Quickborn (DE); Yang XU, Hamburg (DE)

(21) Appl. No.: 18/857,684

(22) PCT Filed: Apr. 12, 2023
(86) PCT No.: PCT/EP2023/059487

§ 371 Date: Oct. 17, 2024

(30) Foreign Application Priority Data
May 12, 2022 (DE) 10 2022 204 661.7

(51) Int. Cl. A61K 8/06; A61K 8/02; A61K 8/34; A61K 8/37; A61K 8/46; A61K 8/60; A61K 8/73; A61K 8/92; A61Q 19/00; A61Q 19/10

(52) U.S. Cl. CPC A61K 8/062; A61K 8/0295; A61K 8/342; A61K 8/375; A61K 8/466; A61K 8/60; A61K 8/604; A61K 8/732; A61K 8/92; A61Q 19/00; A61Q 19/10; A61K 2800/30

(57) ABSTRACT

The invention relates to a cosmetic emulsion.

Cosmetic products generally not only contribute to a beautiful and attractive appearance, but their effects make a crucial contribution to increased self-esteem and people's wellbeing. Accordingly, a wide variety of cosmetic products are used for the daily cleansing and care of human skin.

Skin care products generally consist of emulsions. Emulsions are generally understood to mean heterogeneous systems composed of two liquids that are immiscible, or of only limited miscibility, with each other, which are typically referred to as phases and in which one of the two liquids is dispersed in the other liquid in the form of very fine droplets. Externally and viewed with the naked eye, emulsions appear homogeneous.

If the two liquids are water and oil and oil droplets are present finely distributed in water, it is an oil-in-water emulsion (O/W emulsion, for example milk). The basic character of an O/W emulsion is influenced by the water. The reverse principle applies to a water-in-oil emulsion (W/O emulsion, for example butter), the basic character being determined in this case by the oil.

To stabilize and thicken O/W emulsions, acrylate-based polymers are usually incorporated into these formulations. Acrylate-based polymers are polymers that are obtained from homo- or copolymerization with acrylic and/or methacrylic acid. Examples of these include Carbomer or Acrylates Copolymer.

Emulsions with acrylates are known from documents including DE 10148825 A1, DE 19938756 A1 and DE 29924371 U1.

Acrylate-based polymers are in particular used in jar-compatible emulsions in combination with wax components. What is crucial for these formulations is their rheological properties. For example, the formulations should be viscous enough that they cannot drip out of an open jar under standard conditions. These formulations consequently have only limited flowability under standard conditions. Particularly popular are jar-compatible products that are formulated such that the formulas melt onto the skin after application thereto and thus give the consumer a particular feel on the skin.

However, the use of these acrylate-based polymers is increasingly being subject to criticism since their biodegradability has not been entirely clarified.

What is problematic is the fact that the replacement of carbomers causes the jars to leak particularly easily especially at relatively high temperatures. This can only be compensated for by incorporating relatively high-melting wax components. However, this has the disadvantage that the melting effect on the skin is eliminated. The product benefit is consequently lost.

It was the object of the present invention to provide an emulsion that does not have the disadvantages of the prior art and that allows the melting effect on the skin to be achieved while preventing the product from leaking out at elevated temperatures.

Elevated temperatures are understood to mean temperatures of 45° C., since solar radiation can bring about these temperatures during storage of the products.

Furthermore, DE 2909087 C2 describes a process for determining the consistency of pasty preparations, such as creams. This involves determining the perceptible pressure that arises when a measuring body is guided through the cream in a defined process. The consistency obtained thereby indicates the amount of force that has to be applied in order to guide the measuring body through the cream. This is crucial for products, such as creams, that are stored in jars and the cream is taken up using a finger. It is desirable for formulations to have a sufficient consistency. They can thus be taken up with the finger easily. If the consistency is too low, the finger just moves the cream and it cannot be taken up easily.

A further disadvantage in the prior art is that the consistency of creams and pasty emulsions can often only be kept at a pleasing level by means of acrylate-based polymers. Especially at relatively high temperatures, such as 45° C., emulsions acrylate-free emulsions often have an insufficient consistency.

Surprisingly, the present invention was able to address the problems of the prior art.

The invention provides an acrylate-free cosmetic emulsion comprising
a) an emulsifier system which forms a liquid-crystalline network in the emulsion, the emulsifier system comprising
   a. at least one fatty alcohol glucoside, and
   b. myristyl alcohol,
b) at least one polysaccharide, and
c) at least one wax component having a melting point of less than 43° C.

By definition, acrylate-free means that no polymers obtained from homo-or copolymerization with acrylic and/or methacrylic acid are present.

By definition, fatty alcohols have a C10 to C20 carbon chain. Correspondingly, the alcohol radicals of the fatty alcohol glucosides also have such a carbon chain.

The invention also provides for the use of the emulsion of the invention for providing a cosmetic product.

This is preferably an oil-in-water (O/W) emulsion.

If percentages by weight (% by weight) are stated below without reference to a certain composition or specific mixture, then these figures always relate to the total weight of the cosmetic emulsion. If ratios of components/substances/substance groups are disclosed below, then these ratios relate to weight ratios of the components/substances/substance groups mentioned.

If weight percentage ranges for the constituents of the cosmetic emulsion are stated below, then the disclosure of the present application also encompasses all individual values in steps of 0.1% by weight within these weight percentage ranges.

Within the scope of the present disclosure, the phrases “according to the invention”, “advantageous according to the invention”, “advantageous in the context of the present invention” etc. always relate to both the preparation according to the invention and the use according to the invention.

Unless stated otherwise, all experiments were carried out under standard conditions. The expression “standard conditions” means 20° C., 1013 hPa and a relative humidity of 50%.

If the term skin is used, then this preferably relates to human skin.

If viscosity values are stated in this disclosure, then all values relate to a measurement at 25° C. in a 150 ml wide-neck bottle (VWR No.: 807-001) by means of a Rheomat R 123 from proRheo. The Rheomat R 123 from proRheo GmbH is a rotational viscometer, i.e. a measuring body rotates in the substance to be measured. The force required to rotate the measuring body in the sample at a defined speed is measured. The viscosity is calculated from this torque, the rotational speed of the measuring body and the geometric dimensions of the measuring system used. The measuring body used is the No. 2 measuring body (Article No. 200 0192), speed range 62.5 min⁻¹.

According to the invention, the emulsion has an emulsifier system which forms a liquid-crystalline network in the emulsion. The presence of such a network is correspondingly a feature of the emulsion according to the invention. It is advantageously a liquid-crystalline gel network.

It has surprisingly been found that the gel network specifically formed in this invention, in combination with the further components of the invention, makes it possible to provide acrylate-free, jar-compatible emulsions which do not leak out of the under standard conditions when the jar is tilted after opening, or held upside down. The form of the formulation is maintained. This is also the case at relatively high temperatures, such as 45° C.

Furthermore, it has surprisingly been found that the consistency of the formulation is reduced less at elevated temperatures.

The emulsifier system according to the invention comprises at least one fatty alcohol glucoside. Advantageously, the fatty alcohol glucoside is selected from the group having the INCI names Arachidyl Glucoside; C10-16 Alkyl Glucoside; C12-18 Alkyl Glucoside; C12-20 Alkyl Glucoside; Coco-glucoside; C9-11 Alkyl Glucoside; Caprylyl/Capryl Glucoside; Caprylyl Glucoside; Cetearyl Glucoside; Decyl Glucoside; Isostearyl Glucoside; Lauryl Glucoside; Myristyl Glucoside and/or Undecyl Glucoside. Preference is given to Cetearyl Glucoside, Caprylyl Glucoside, Caprylyl/Capryl Glucoside, Lauryl Glucoside and Myristyl Glucoside. Cetearyl Glucoside is especially preferred.

Advantageously, the proportion by weight of the entirety of the fatty alcohol glucosides in the emulsion is from 0.1% to 5.0% by weight, preferably from 0.8% to 3.5% by weight, preferably 1.0% to 2.5% by weight and especially preferably from 1.2% to 2.2% by weight, in each case based on the total weight of the emulsion.

It is also advantageous if the total proportion by weight of Arachidyl Glucoside; C10-16 Alkyl Glucoside; C12-18 Alkyl Glucoside; C12-20 Alkyl Glucoside; Coco-glucoside; C9-11 Alkyl Glucoside; Caprylyl/Capryl Glucoside; Caprylyl Glucoside; Cetearyl Glucoside; Decyl Glucoside; Isostearyl Glucoside; Lauryl Glucoside; Myristyl Glucoside and/or Undecyl Glucoside is from 0.1% to 5.0% by weight, preferably from 0.8% to 3.5% by weight, preferably 1.0% to 2.5% by weight and especially preferably from 1.2% to 2.2% by weight, in each case based on the total weight of the emulsion.

It is especially advantageous if the proportion of Cetearyl Glucoside, Caprylyl Glucoside, Caprylyl/Capryl Glucoside, Lauryl Glucoside and/or Myristyl Glucoside is from 0.1% to 5.0% by weight, preferably from 0.8% to 3.5% by weight, preferably 1.0% to 2.5% by weight and especially preferably from 1.2% to 2.2% by weight, in each case based on the total weight of the emulsion.

It is especially advantageous if the proportion of Cetearyl Glucoside is from 0.1% to 5.0% by weight, preferably from 0.8% to 3.5% by weight, preferably 1.0% to 2.5% by weight and especially preferably from 1.2% to 2.2% by weight, in each case based on the total weight of the emulsion.

Furthermore, it is advantageous if the emulsifier system comprises Sorbitan Stearate. If Sorbitan Stearate is present, the total proportion of Sorbitan Stearate is advantageously from 0.1% to 1.5% by weight, preferably 0.15% to 1.0% by weight and in particular from 0.2% to 0.8% by weight, based on the total weight of the emulsion.

Furthermore, it is advantageous if the emulsifier system comprises Disodium Cetearyl Sulfosuccinate. If Disodium Cetearyl Sulfosuccinate is present, the total proportion of Disodium Cetearyl Sulfosuccinate is advantageously from 0.01% to 0.5% by weight, preferably 0.02% to 0.2% by weight and in particular from 0.04% to 0.10% by weight, based on the total weight of the emulsion.

Furthermore, it is advantageous if the emulsifier system comprises Glyceryl Stearate. If Glyceryl Stearate is present, the total proportion of Glyceryl Stearate is advantageously from 0.1% to 1.8% by weight, preferably 0.5% to 1.5% by weight and in particular from 0.7% to 1.2% by weight, based on the total weight of the emulsion.

Furthermore, the emulsifier system additionally comprises myristyl alcohol. Advantageously, the proportion of the myristyl alcohol is from 0.2% to 6.5% by weight, preferably 0.5% to 6.0% by weight, preferably 1.5% to 5.0% by weight, preferably 2.5% to 4.5% by weight and especially preferably 3.0% to 4.0% by weight, in each case based on the total weight of the emulsion.

It was in fact surprising to those skilled in the art that it is possible to form liquid-crystalline structures with a fatty alcohol that has a melting point of less than 40° C. Usually, it is necessary to use fatty alcohols having a higher melting point, such as Stearyl Alcohol. Surprisingly, the further components of the invention in combination with myristyl alcohol allow the formation of the liquid-crystalline structures.

Liquid-crystalline structures in emulsions can be visualized by means of light microscopy with polarized light. Reference is made to the publication “Study on the Formation and Properties of Liquid Crystal Emulsion in Cosmetic”, Wanping Zhang*, Lingyan Liu, School of Perfume and Aroma technology, Shanghai Institute of Technology, Shanghai, China. Journal of Cosmetics, Dermatological Sciences and Applications, 2013.

Advantageously, the liquid-crystalline structures in the emulsion of the invention are present in globular structure. This is identifiable by the appearance of the typical Maltese crosses in light microscopy images with polarized light.

The emulsion of the invention also comprises at least one polysaccharide. Advantageously, the polysaccharides are selected from the groups of the polysaccharide gums, the starches and/or celluloses.

Among the polysaccharide gums, use is preferably made in particular of the substances known under the INCI names Xanthan Gum, Guar Gum, Gellan Gum, Sclerotium Gum and/or Tara Gum. Xanthan Gum, Guar Gum and/or Gellan Gum are especially preferred.

Among the starches, use is preferably made of the substances known under the INCI names Zea Mays Starch, Sodium Hydroxypropyl Starch Phosphate, Sodium Carboxymethyl Starch, Hydroxypropyl Starch Phosphate, Tapioca Starch, Potato Starch Modified and/or Distarch Phosphate. Use is especially preferably made of nonionic starches, in particular Hydroxypropyl Starch Phosphate and/or Distarch Phosphate, with Hydroxypropyl Starch Phosphate being the most preferred.

Among the celluloses, use is preferably made of the substances known under the INCI names Ethylcellulose, Microcrystalline Cellulose, Cellulose Gum, Hydroxyethylcellulose, Carboxymethyl Hydroxyethylcellulose, Hydroxyethyl Ethylcellulose, Hydroxymethyl Cellulose, Hydroxypropylcellulose, Hydroxypropyl Methylcellulose and/or Hydroxyethylcellulose.

Advantageously, the proportion of the polysaccharides in the emulsion is from 0.05% to 4% by weight, preferably 0.1% to 2.5% by weight, preferably from 0.2% to 1.5% by weight and especially preferably from 0.3% to 1.0% by weight, based on the total weight of the emulsion. Advantageously, the proportion of the polysaccharide gums in the emulsion is from 0.1% to 4% by weight, preferably 0.2% to 2.5% by weight, preferably from 0.3% to 1.5% by weight and especially preferably from 0.4% to 1.0% by weight, based on the total weight of the emulsion.

Advantageously, the total proportion of Xanthan Gum, Guar Gum and/or Gellan Gum in the emulsion is from 0.01% to 4% by weight, preferably 0.05% to 2.5% by weight, preferably from 0.07% to 1.5% by weight and especially preferably from 0.1% to 1.0% by weight, based on the total weight of the emulsion.

Advantageously, the proportion of the starches in the emulsion is from 0.05% to 4% by weight, preferably 0.1% to 2.5% by weight, preferably from 0.15% to 1.5% by weight and especially preferably from 0.2% to 1.0% by weight, based on the total weight of the emulsion.

Advantageously, the total proportion of Zea Mays Starch, Sodium Hydroxypropyl Starch Phosphate, Sodium Carboxymethyl Starch, Hydroxypropyl Starch Phosphate, Tapioca Starch, Potato Starch Modified and/or Distarch Phosphate in the emulsion is from 0.05% to 4% by weight, preferably 0.1% to 2.5% by weight, preferably from 0.15% to 1.5% by weight and especially preferably from 0.2% to 1.0% by weight, based on the total weight of the emulsion.

Advantageously, the total proportion of Hydroxypropyl Starch Phosphate and/or Distarch Phosphate in the emulsion is from 0.05% to 4% by weight, preferably 0.1% to 2.5% by weight, preferably from 0.15% to 1.5% by weight and especially preferably from 0.2% to 1.0% by weight, based on the total weight of the emulsion.

Advantageously, the total proportion of Hydroxypropyl Starch Phosphate in the emulsion is from 0.05% to 4% by weight, preferably 0.1% to 2.5% by weight, preferably from 0.15% to 1.5% by weight and especially preferably from 0.2% to 1.0% by weight, based on the total weight of the emulsion.

Advantageously, the proportion of the celluloses in the emulsion is from 0.05% to 4% by weight, preferably 0.1% to 2.5% by weight, preferably from 0.2% to 1.5% by weight and especially preferably from 0.3% to 1.0% by weight, based on the total weight of the emulsion. Advantageously, the total proportion of Ethylcellulose, Microcrystalline Cellulose, Cellulose Gum, Hydroxyethylcellulose, Carboxymethyl Hydroxyethylcellulose, Hydroxyethyl Ethylcellulose, Hydroxymethyl Cellulose, Hydroxypropylcellulose, Hydroxypropyl Methylcellulose and/or Hydroxyethylcellulose in the emulsion is from 0.1% to 4% by weight, preferably 0.05% to 4% by weight, preferably 0.1% to 2.5% by weight, preferably from 0.2% to 1.5% by weight and especially preferably from 0.3% to 1.0% by weight, based on the total weight of the emulsion.

Furthermore, the emulsion comprises at least one wax component having a melting point of less than 43° C.

Advantageously, at least one wax component having a melting point in the range from 25° C. to 43° C. is present. The presence of such low-melting wax components ensures the melting of the preparation on human skin. Surprisingly, it was simultaneously possible to better maintain the consistency (less reduced), in particular at temperatures around 45° C. In addition, it was surprisingly possible to prevent phase separations at 45° C.

Fatty alcohols and fatty acids having alkyl chains with 10 to 20 carbon atoms by definition are not considered to be wax components.

Advantageously, the total content of the wax components having a melting point in the range from 25° C. to 43° C. is from 0.5% to 5% by weight, preferably from 1.5% to 4.5% by weight and especially preferably 2.2% to 3.5% by weight, based on the total weight of the emulsion.

Preferred wax components in the stated melting range are Myristyl Myristate, Hydrogenated Coco-Glycerides and/or Cetyl Ricinoleate.

Advantageously, the total content of Myristyl Myristate, Hydrogenated Coco-Glycerides and/or Cetyl Ricinoleate is from 0.5% to 5% by weight, preferably 1.5% to 4.5% by weight and especially preferably 2.2% to 3.5% by weight, based on the total weight of the emulsion.

If Myristyl Myristate is present, it is advantageous if the proportion of Myristyl Myristate is from 0.5% to 5.5% by weight, preferably 1.0% to 4.5% by weight and especially preferably from 1.2% to 3.0% by weight, based on the total weight of the emulsion. If Hydrogenated Coco-Glycerides is present, it is preferred if the proportion of Hydrogenated Coco-Glycerides is from 0.1% to 3.0% by weight, preferably 0.3% to 2.0% by weight and preferably 0.5% to 1.5% by weight, based on the total weight of the emulsion. If Cetyl Ricinoleate is present, it is preferred if the proportion of Cetyl Ricinoleate is from 0.01% to 3.0% by weight, preferably 0.05% to 1.0% by weight and preferably 0.1% to 0.5% by weight, based on the total weight of the emulsion.

Furthermore, it is advantageous according to the invention if high-melting waxes having a melting point of more than 43° C. are not present, or the proportions thereof are less than 0.5% by weight of the emulsion. Surprisingly, it was possible to provide stable formulations at 45° C., without the need to incorporate high-melting wax components. It is assumed that the formation of the liquid-crystalline structures at 45° C. enables the stability of the formulation to prevent phase separations to maintain the consistency.

Furthermore, it is advantageous if the emulsion comprises one or more oil components that are liquid under standard conditions. In general, it is preferred if the proportion of the oil components that are liquid under standard conditions is from 0.2% to 5.0% by weight, preferably 0.5% to 4.0% by weight and especially preferably 1.5% to 2.5% by weight, based on the total weight of the emulsion.

Oils that are advantageously present are selected from natural oils. Natural oils that are advantageously present are selected from the group of Persea Gratissima Oil, Orbignya Oleifera Seed Oil, Argania Spinosa Kernel Oil, Prunus Armeniaca Kernel Oil, Simmondsia Chinensis Seed Oil, Butyrospermum Parkii Butter, Cocos Nucifera Oil, Silybum Marianum Seed Oil, Oenothera Biennis Oil, Olea Europaea Fruit Oil, Helianthus Annuus Seed Oil, Vitis Vinifera Seed Oil, Cannabis Sativa Seed Oil, Vegetable Oil, Gossypium Herbaceum Seed Oil, Arctium Lappa Seed Oil, Macadamia Ternifolia Seed Oil, Macadamia Integrifolia Seed Oil, Zea Mays Germ Oil, Prunus Amygdalus Dulcis Oil, Ricinus Communis Seed Oil, Brassica Campestris Seed Oil and/or Glycine Soja Oil. If natural oils are present, the total content of these oils is advantageously from 0.01% to 3% by weight, preferably 0.1% to 1.0% by weight and especially preferably from 0.2% to 0.8% by weight, based on the total weight of the emulsion.

Furthermore, it is advantageous if one or more esters of coconut fatty alcohol are present. Use is preferably made here in particular of Coco-Caprylate/Caprate. If an ester of a coconut fatty alcohol is present, the proportion of this ester is advantageously from 0.2% to 4% by weight, preferably 0.4% to 2.5% by weight and especially preferably from 0.8% to 1.5% by weight, based on the total weight of the emulsion. If Coco-Caprylate/Caprate is present, the proportion of Coco-Caprylate/Caprate is advantageously from 0.2% to 4% by weight, preferably 0.4% to 2.5% by weight and especially preferably from 0.8% to 1.5% by weight, based on the total weight of the emulsion.

Furthermore, it is preferred if one or more oils that are triglycerides of 3 identical fatty acids are present. Triisostearin is especially preferred. If triglycerides of 3 identical fatty acids are present, the proportion of these triglycerides is advantageously from 0.05% to 2% by weight, preferably 0.08% to 1.0% by weight and especially preferably from 0.1% to 0.5% by weight, based on the total weight of the emulsion. If Triisostearin is present, the proportion of Triisostearin is advantageously from 0.05% to 2% by weight, preferably 0.08% to 1.0% by weight and especially preferably from 0.1% to 0.5% by weight, based on the total weight of the emulsion.

Additionally, it is advantageous if one or more fatty alcohols having 16 to 24 carbon atoms are present, with the total proportion of these fatty alcohols being from 0.01% to 1.5% by weight, preferably from 0.05% to 0.3% by weight, based on the total weight of the emulsion. In particular, cetearyl alcohol is present at a proportion of 0.01% to 1.0% by weight, preferably from 0.05% to 0.3% by weight, based on the total weight of emulsion.

Furthermore, it has been shown to be advantageous if one or more fatty acids having 14 to 24 carbon atoms are present in the emulsion, with the proportion thereof advantageously being from 0.2% to 5.0% by weight, preferably from 1.5% by weight to 4% by weight and especially preferably from 1.8% to 3.0% by weight, based on the total weight of the emulsion.

The emulsion according to the invention is also preferably characterized in that this emulsion comprises glycerol at a proportion of 0.5% by weight to 15% by weight, preferably 3.0% to 7% by weight, based on the total weight of the emulsion.

Furthermore, it is also advantageous in the context of the present invention if the emulsion comprises phenoxyethanol, dehydroacetic acid, benzyl alcohol and/or ethylhexylglycerin.

If the emulsion comprises benzyl alcohol, it is preferred if the proportion of benzyl alcohol is from 0.05% by weight to 0.5% by weight, based on the total weight of the emulsion.

If the emulsion comprises phenoxyethanol, it is preferred if the total proportion of phenoxyethanol is from 0.1% by weight to 1.2% by weight, based on the total weight of the emulsion.

If the emulsion comprises ethylhexylglycerin, it is preferred if the proportion of ethylhexylglycerin is from 0.1% by weight to 1.0% by weight, based on the total weight of the emulsion.

Moreover, it is advantageous if embodiments of the invention are characterized in that they comprise ethanol. If ethanol is present in the emulsion, the proportion of ethanol is preferably from 0.5% by weight to 10% by weight, preferably 1.5% by weight to 5.0% by weight, based on the total weight of the emulsion.

Last but not least, embodiments that are advantageous according to the invention are characterized in that the emulsion comprises one or more active ingredients selected from the group of the compounds glycyrrhetinic acid, urea, arctiin, folic acid, coenzyme Q10 (ubiquinone), alpha-glucosylrutin, carnitine, carnosine, caffeine, natural and/or synthetic isoflavonoids, glycerylglucose, creatine, creatinine, taurine, tocopherol, tocopherol acetate, vitamin C, vitamin C phosphate, vitamin C palmitate, niacinamide, vitamin A palmitate, panthenol, licochalcone A, rucinol, N-[(2,4-dihydroxyphenyl)thiazol-2-yl]isobutyramide, honokiol and magnolol (also as constituent of magnolia extracts), hyaluronic acid and/or silymarin (milk thistle extract).

Emulsions that are advantageous according to the invention are also characterized in that they comprise water at a proportion of 60% by weight to 95% by weight and preferably from 70% by weight to 90% by weight, based on the total weight of the emulsion.

Furthermore, emulsions that are advantageous according to the invention have a viscosity 24 h after preparation of 9000 mPa·s to 30 000 mPa·s, advantageously 12 000 mPa·s to 20 000 mPa·s. If viscosity is referred to in this disclosure, then all values relate to a measurement at 25° C. in a 150 ml rolled rim glass bottle by means of a Rheomat R 123 from proRheo. The Rheomat R 123 from proRheo GmbH is a rotational viscometer, i.e. a measuring body rotates in the substance to be measured. The force required to rotate the measuring body in the sample at a defined speed is measured. The viscosity is calculated from this torque, the rotational speed of the measuring body and the geometric dimensions of the measuring system used. The measuring body used is the No. 2 measuring body (Article No. 200 0192), speed range 62.5 min⁻¹. All measurements for viscosity are always performed 24 h after preparation of the emulsion.

### COMPARATIVE EXPERIMENTS AND EXAMPLES

The examples below are intended to illustrate the present invention without limiting it. Unless stated otherwise, all amounts, proportions and percentages are based on the weight and the total amount or on the total weight of the preparations.

The table below with Ex. 1 to Ex. 8 shows different formulations. Ex. 1 is a non-inventive example that represents a customary formulation having the acrylate-based polymer Carbomer. The formulation does not have any liquid-crystalline structures. The formulation is stable against water separations when stored at both 40° C. and 45° C. for 8 months. In addition, this formulation also has a pleasing consistency even at high temperatures of 45° C.

As shown by non-inventive Examples 2 to 4, the replacement of acrylate-based polymers with a biopolymer results in the formulations having an insufficient stability against water separations and also having a low consistency at 45° C. These examples do not exhibit any liquid-crystalline structures.

Examples 5 to 7 are examples according to the invention. These have liquid-crystalline structures, which were demonstrated by means of light microscopy with polarized light. Surprisingly, it has been found that the presence of the liquid-crystalline structures leads to a stabilization of the formulations, with the result that water separations are prevented at elevated temperatures in the case of storage for 8 months. In addition, a significantly increased consistency was surprisingly determined at 45° C. Consequently, the disadvantages caused by dispensing with acrylate-containing polymers were mitigated by the present invention.

The consistency was measured by means of the process described in DE 2909087 C2.

An 8 mm polyamide ball as measuring instrument is drawn once along a circuit through the test emulsion under defined conditions (at uniform speed and constant maximum immersion depth). The cream consistency-dependent braking force is measured using a leaf spring test stand, the ball being fastened to the sensor thereof. Since the ball is drawn through each zone of the cream only once, influencing factors, for example due to thixotropic properties and possible slide-flow effects, are largely avoided. The speed of the rotation axle is set to 10 rpm. The maximum braking force acting on the ball is determined. It is crucial that the ball is immersed to the same depth and distance in all measurements. The determined values are scale values, with one scale value corresponding to 1 cN. The scale values are reported in the table with the formulations. The sample temperature was 45° C.

Furthermore, an optical evaluation was performed for the examples, as reported in the table below. Furthermore, the storage modulus G′ of the formulations Ex. 1, Ex. 2 and Ex. 6 was determined. The storage modulus provides information about the amount of structure present in a material. It represents the energy stored in the elastic structure of the sample.

The following measurement parameters were selected:
Instrument: ARES 6
Method: SOF (soften, DynamicTemperatureRamp)
Temperature: 20° C. to 80° C. with 2° C./min
Geometry: Parallel plates 50 mm and 1.0 mm gap
Frequency: 100 rad s⁻¹

The measurement results at 20° C. and 45° C. are listed in the following table:

| Samples | Storage modulus [Pa] @ 20° C. | Storage modulus [Pa] @ 45° C. | $\frac{{G}^{′} @ 45 ° C .}{{G}^{′} @ 20 ° C .} \times 100 \%$ |
|---|---|---|---|
| Ex. 1 | 2900 | 1000 | 34% |
| Ex. 2 | 1700 | 400 | 23% |
| Ex. 6 | 2000 | 800 | 40% |

When the storage moduli at 20° C. and 45° C. for the respective samples are compared, it is noticeable that at 45° C. Ex. 6 still has a similar amount (approx. 40%) of its storage modulus in comparison with the value at 20° C. This is comparable to the values for Ex. 1, which contains an acrylate-containing polymer. Ex. 2 cannot maintain the same values as Ex. 1 at 45° C.

| INCI | | Ex. 1 | Ex. 2 | Ex. 3 | Ex. 4 | Ex. 5 | Ex. 6 | Ex. 7 |
|---|---|---|---|---|---|---|---|---|
| Vegetable Oil | | 0.3 | 0.3 | 0.3 | 0.3 | 0.3 | 0.3 | 0.3 |
| Coco-Caprylate/Caprate | | 1.2 | 1.2 | 1.2 | 1.2 | 1.2 | 1.2 | 1.2 |
| Simmondsia Chinensis Seed Oil | | 0.1 | 0.1 | 0.1 | 0.1 | 0.1 | 0.1 | 0.1 |
| Prunus Amygdalus Dulcis Oil | | 0.1 | 0.1 | 0.1 | 0.1 | 0.1 | 0.1 | 0.1 |
| Triisostearin | | 0.3 | 0.3 | 0.3 | 0.3 | 0.3 | 0.3 | 0.3 |
| Myristyl Alcohol | | 3.5 | 3.5 | 3.5 | 2.5 | 3.5 | 3.5 | 3.5 |
| Hydrogenated Coco-Glycerides | | 1 | 1 | 1 | 1 | 1 | 1 | 1 |
| Myristyl Myristate | | 1.5 | 1.5 | 1.5 | 1.5 | 1.5 | 1.5 | 1.5 |
| Cetyl Ricinoleate | | 0.1 | 0.1 | 0.1 | 0.1 | 0.1 | 0.1 | 0.1 |
| Cetearyl Alcohol | | 0.1 | 0.1 | 0.1 | 0.1 | 0.1 | 0.1 | 0.1 |
| Glyceryl Stearate | | 1.1 | 1.1 | 1.1 | 1.1 | 1.1 | 1.1 | 1.1 |
| Palmitic Acid | | 1.2 | 1.2 | 1.2 | 1.2 | 1.2 | 1.2 | 1.2 |
| Stearic Acid | | 1.2 | 1.2 | 1.2 | 1.2 | 1.2 | 1.2 | 1.2 |
| Glyceryl Stearate Citrate | | 1.5 | 1.5 | 1.5 | 0.1 | | | |
| Polyglyceryl-3 Methylglucose | | | | 0.8 | | | | |
| Distearate | | | | | | | | |
| Polyglyceryl-3 Distearate | | | | | 2.5 | | | |
| Cetearyl Glucoside | | | | | | 1.8 | 1.5 | 1.0 |
| Sorbitan Stearate | | | | | | 0.8 | 0.7 | 0.4 |
| Disodium Cetearyl | | | | | | 0.1 | 0.1 | 0.1 |
| Sulfosuccinate | | | | | | | | |
| Hydroxypropyl Starch | | 0 | 0.8 | 0.8 | 0.8 | 0.8 | 0.8 | 0.8 |
| Phosphate | | | | | | | | |
| Carbomer | | 0.3 | 0 | 0 | 0 | 0 | 0 | 0 |
| Perfume | | 0.2 | 0.2 | 0.2 | 0.2 | 0.2 | 0.2 | 0.2 |
| Glycerol | | 4.5 | 4.5 | 4.5 | 4.5 | 4.5 | 4.5 | 4.5 |
| Sodium Hydroxide | | q.s. | q.s. | q.s. | q.s. | q.s. | q.s. | q.s. |
| Phenoxyethanol | | 0.9 | 0.9 | 0.9 | 0.9 | 0.9 | 0.9 | 0.9 |
| Alcohol Denat. | | 3 | 3 | 3 | 3 | 3 | 3 | 3 |
| Aqua | | Ad 100 | Ad 100 | Ad 100 | Ad 100 | Ad 100 | Ad 100 | Ad 100 |
| Stability 8 months (water | 40° C. | 100 | 20 | 40 | 60 | 100 | 100 | 100 |
| separation, 100% = no | 45° C. | 100 | 0 | 20 | 20 | 100 | 100 | 100 |
| water separation) | | | | | | | | |
| Consistency scale value | | 14 | 2 | 2 | 3 | 12 | 11 | 7 |
| at 45° C. | | | | | | | | |
| Optical evaluation comparing | | No | Formula | Formula | Formula | No | No | No |
| 45° C. samples to samples | | movement | immediately | immediately | immediately | movement | movement | movement |
| at 20° C. (visual | | of the | runs | runs | runs | of the | of the | of the |
| assessment of the movement | | formula | downward, | downward, | downward, | formula | formula | formula |
| of the cream in a 30 ml | | | behavior | behavior | behavior | | | |
| glass bottle when the bottle | | | like water | like water | like water | | | |
| is placed upside down) | | | | | | | | |

### Further Examples

| INCI | Ex. A | Ex. B | Ex. C | Ex. D | Ex. E | Ex. F |
|---|---|---|---|---|---|---|
| Tocopherol | 0.1 | 0 | 0 | 0.2 | 0 | 0 |
| Vegetable Oil | 0 | 0.3 | 0 | 0 | 0 | 0.4 |
| Coco-Caprylate/Caprate | 0 | 0 | 1 | 0 | 0 | 0 |
| Ethylhexyl Cocoate | 1.0 | 0 | 0 | 0 | 0 | 0 |
| Octyldodecanol | 0 | 1 | 0 | 0 | 0.6 | 0 |
| Simmondsia Chinensis Seed Oil | 0 | 0 | 0.1 | 0 | 0 | 0 |
| Butyrospermum Parkii Butter | 0.1 | 0.5 | 0 | 0.4 | 0.7 | 0 |
| Glycine Soja Oil | 0 | 0 | 0.5 | 0 | 0 | 0 |
| Prunus Amygdalus Dulcis Oil | 0 | 0 | 0 | 0 | 0 | 0.2 |
| Helianthus Annuus Seed Oil | 0 | 0 | 0.1 | 1.5 | 0 | 0.5 |
| Brassica Campestris Seed Oil | 0 | 0 | 0 | 0 | 0.2 | 0 |
| Triisostearin | 0 | 0.1 | 0 | 0 | 0.3 | 0 |
| Myristyl Alcohol | 5 | 4 | 3 | 3 | 5 | 5 |
| Hydrogenated Coco-Glycerides | 0.7 | 0.6 | 1 | 1.5 | 1.3 | 1 |
| Myristyl Myristate | 1 | 1.2 | 1.5 | 1 | 0.8 | 0.7 |
| Cetyl Ricinoleate | 0.1 | 0.1 | 0.3 | 0.2 | 0.1 | 0.4 |
| Cetearyl Alcohol | 0.2 | 1 | 0.1 | 1.5 | 0.1 | 0.2 |
| Cetyl Palmitate | 0 | 0 | 0 | 0.2 | 0 | 0 |
| Glyceryl Stearate | 1.3 | 0.7 | 1.2 | 0.8 | 1.1 | 0.7 |
| Palmitic Acid | 1.5 | 1 | 1.3 | 0.8 | 1.1 | 1 |
| Stearic Acid | 1.5 | 1 | 1.3 | 0.8 | 1.1 | 1 |
| Cetearyl Glucoside | 0.9 | 1 | 1.6 | 1.5 | 0.7 | 2.2 |
| Sorbitan Stearate | 0.2 | 0.3 | 0.7 | 0.1 | 0.5 | 1.2 |
| Disodium Cetearyl | 0.1 | 0 | 0.1 | 0 | 0 | 0 |
| Sulfosuccinate | | | | | | |
| Hydroxypropyl Starch Phosphate | 0.5 | 0.4 | 0.6 | 0 | 0.8 | 0 |
| Distarch Phosphate | 0 | 0.4 | 0 | 1 | 0 | 2 |
| Gellan Gum | 0.2 | 0 | 0 | 0.1 | 0.1 | 0 |
| Xanthan Gum | 0 | 0 | 0.1 | 0 | 0 | 0 |
| Perfume | 0.1 | 0.3 | 0.3 | 0.2 | 0.6 | 0.4 |
| Glycerol | 3 | 5 | 3.5 | 10 | 4 | 7 |
| Sodium Hydroxide | q.s. | q.s. | q.s. | q.s. | q.s. | q.s. |
| Phenoxyethanol | 0.7 | 0.9 | 0 | 0.8 | 0.3 | 0.4 |
| Ethylhexylglycerin | 0.1 | 0 | 0.3 | 0 | 0.2 | 0.2 |
| Hydroxyacetophenone | 0 | 0 | 0.1 | 0 | 0 | 0.2 |
| Benzyl alcohol | 0 | 0 | 0.3 | 0.1 | 0.1 | 0 |
| Alcohol Denat. | 3 | 4 | 5 | 3 | 5 | 5 |
| Aqua | Ad 100 | Ad 100 | Ad 100 | Ad 100 | Ad 100 | Ad 100 |

1.-15. (canceled)

16. A cosmetic emulsion, wherein the emulsion is acrylate-free and comprises
(a) an emulsifier system which forms a liquid-crystalline network in the emulsion and comprises
(i) at least one fatty alcohol glucoside, and
(ii) myristyl alcohol,
(b) at least one polysaccharide, and
(c) at least one wax component having a melting point of less than 43° C.

17. The emulsion of claim 16, wherein the emulsion is an oil-in-water emulsion.

18. The emulsion of claim 16, wherein the at least one fatty alcohol glucoside comprises one or more compounds with the INCI names Arachidyl Glucoside; C10-16 Alkyl Glucoside; C12-18 Alkyl Glucoside; C12-20 Alkyl Glucoside; Coco-glucoside; C9-11 Alkyl Glucoside; Caprylyl/Capryl Glucoside; Caprylyl Glucoside; Cetearyl Glucoside; Decyl Glucoside; Isostearyl Glucoside; Lauryl Glucoside; Myristyl Glucoside; Undecyl Glucoside.

19. The emulsion of claim 16, wherein the at least one fatty acid glucoside comprises one or more of Cetearyl Glucoside, Caprylyl Glucoside, Caprylyl/Capryl Glucoside, Lauryl Glucoside, Myristyl Glucoside.

20. The emulsion of claim 18, wherein the at least one fatty acid glucoside comprises Cetearyl Glucoside.

21. The emulsion of claim 16, wherein the emulsion comprises from 0.1% to 5.0% by weight of the one or more fatty acid glucosides, based on a total weight of the emulsion.

22. The emulsion of claim 18, wherein the emulsion comprises from 1.2% to 2.2% by weight of the one or more fatty acid glucosides, based on a total weight of the emulsion.

23. The emulsion of claim 16, wherein the emulsion comprises from 0.1% to 5.0% by weight of cetearyl glucoside, based on a total weight of the emulsion.

24. The emulsion of claim 16, wherein the emulsion comprises sorbitan stearate.

25. The emulsion of claim 16, wherein the emulsion comprises Disodium Cetearyl Sulfosuccinate.

26. The emulsion of claim 16, wherein the emulsion comprises Glyceryl Stearate.

27. The emulsion of claim 16, wherein the emulsion comprises from 0.2% to 6.5% by weight of myristyl alcohol, based on a total weight of the emulsion.

28. The emulsion of claim 16, wherein the emulsion comprises from 3.0% to 4.0% by weight of myristyl alcohol, based on a total weight of the emulsion.

29. The emulsion of claim 16, wherein liquid-crystalline structures in the emulsion are present as globular structures.

30. The emulsion of claim 16, wherein the at least one polysaccharide comprises one or more of a polysaccharide gum, a starch, and a cellulose.

31. The emulsion of claim 16, wherein the at least one polysaccharide comprises one or more of Zea Mays Starch, Sodium Hydroxypropyl Starch Phosphate, Sodium Carboxymethyl Starch, Hydroxypropyl Starch Phosphate, Tapioca Starch, Potato Starch Modified, Distarch Phosphate.

32. The emulsion of claim 16, wherein the at least one polysaccharide comprises one or more of Hydroxypropyl Starch Phosphate and Distarch Phosphate.

33. The emulsion of claim 16, wherein the emulsion comprises from 0.05% to 4% by weight of at least one starch, based on a total weight of the emulsion.

34. The emulsion of claim 16, wherein the emulsion comprises from 0.5% to 5% by weight of at least one wax component having a melting point of from 25° C. to 43° C., based on a total weight of the emulsion.

35. The emulsion of claim 16, wherein the emulsion comprises from 2.2% to 3.5% by weight of at least one wax component having a melting point of from 25° C. to 43° C., based on a total weight of the emulsion.
